(19) United States
(12) Patent Application Publication
(10) Pub. No.: US 2011/0054389 A1
(43) Pub. Date: Mar. 3, 2011

(54) Method and apparatus for cleaning a nasal passage

(76) Inventor: Christopher Do, San Jose, CA (US)

(21) Appl. No.: 12/584,167

(22) Filed: Aug. 31, 2009

(51) Int. Cl. A61M 1/00; A61M 3/00

(52) U.S. Cl. 604/28; 604/35; 604/30

(57) ABSTRACT

A portable device for cleaning a nasal passage is disclosed that includes: a first receptacle having a first opening and a first stop, a second receptacle, a power driven motor operable to deliver an adjustably continuous flushing pressure to the first receptacle and to provide an adjustably continuous vacuuming pressure to second receptacle. The portable device also includes a first pressure adjusting means operable to control the amount of the flushing pressure, and a second pressure adjusting means, operable to control the amount of the vacuuming pressure.

### FIELD OF THE INVENTION

The present invention relates generally to the field of medical devices. More specifically, the present invention relates to nasal cleaning device for home use.

### BACKGROUND ART

Children below the age of three often experience chronic nasal congestion and drainage and they cannot clear it themselves. Leaving congested nasal passages unchecked leads to restricted breathing, lower oxygen levels, increased stress on the lungs and vascular system. In addition, congested nasal passages may cause overall discomfort for children and anxieties for parents.

Traditionally, parents use a rubber suction device with a resilient rubber cavity and an integral nipple to remove mucus from a child's nasal passage. One problem with these devices is that they do not provide continuous suction and they can be clogged up very quickly, rendering this device temporarily useless until unclogging. Another problem is that they cannot remove solidified or thick mucus. Yet another problem is that the rubber suction device is difficult to use for children because children do not like to have a foreign object inserted into their nasal passages. They would cry and turn their head away, rendering the prior art suction device ineffective and dangerous to children. During the suction of mucus, a child may suddenly turn their body or their head, possibly causing the nipple to accidentally intrude deep into their nasal passage. This would cause discomfort and sometimes injuries to a child's sensitive nasal passage. As a result, children may react strongly and refuse the use of such suction device on them the next time they have a nasal passage congestion or drainage.

Yet another problem of the rubber suction device is that it cannot completely clean a child's nasal passage. This is because the rubber suction device is designed to remove a mucus clog rather than to completely clean a child's nasal passage. In order to completely clean the child's nasal passage, parents often use a wet cotton tip that intrudes deeply thereto, further causing discomfort to the child.

Parents cannot take their children to a hospital for more sophisticated devices for clearing children's nasal congestion and drainage problems. In other words, sophisticated nasal passage clearing devices are not available to parents at home for their convenience.

Therefore what is needed is an effective home device for clearing a nasal passage that does not cause the above described problems.

### SUMMARY OF THE INVENTION

Accordingly, an objective of the present invention is to provide a portable device that can efficiently and conveniently remove and clean a nasal passage. Thus, portable device for cleaning a nasal passage is disclosed that includes: a first receptacle having a first opening and a first stop, a second receptacle, a power driven motor operable to deliver an adjustably continuous flushing pressure to the first receptacle and to provide an adjustably continuous vacuuming pressure to second receptacle. The portable device also includes a first pressure adjusting means operable to control the amount of the flushing pressure, and a second pressure adjusting means, operable to control the amount of the vacuuming pressure.

These and other advantages of the present invention will no doubt become obvious to those of ordinary skill in the art after having read the following detailed description of the preferred embodiments, which are illustrated in the various drawing Figures.

### BRIEF DESCRIPTION OF THE DRAWINGS

The accompanying drawings, which are incorporated in and form a part of this specification, illustrate embodiments of the invention and, together with the description, serve to explain the principles of the invention.

FIG. 1 is a diagram illustrating a perspective view of a nasal clearing device;

FIG. 2 is a diagram illustrating a cutaway view of the nasal clearing device from FIG. 1;

FIG. 3 is a diagram illustrating a cross-sectional view of one embodiment of a saline reservoir for use with a nasal clearing device;

FIG. 4 is a diagram illustrating a cross-sectional view of one embodiment of a bulb for use with a nasal clearing device;

FIG. 5. is a diagram illustrating one embodiment a bulb for use with a nasal clearing device;

FIG. 6 is a block diagram illustrating pressure flow for the nasal clearing device of FIG. 1; and

FIG. 7 is a diagram illustrating a perspective view of one embodiment of a bulb and application thereof to a child's nose;

FIG. 8 is a schematic diagram illustrating all components of the portable nasal passage cleaning device in accordance with an embodiment of the present invention;

FIG. 9 is a flow chart illustrating the process of cleaning and removing secretions of a congested nasal passage in accordance with an embodiment of the present invention.

### DETAILED DESCRIPTION OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well-known methods, procedures, components, and circuits have not been described in detail so as not to unnecessarily obscure aspects of the present invention.

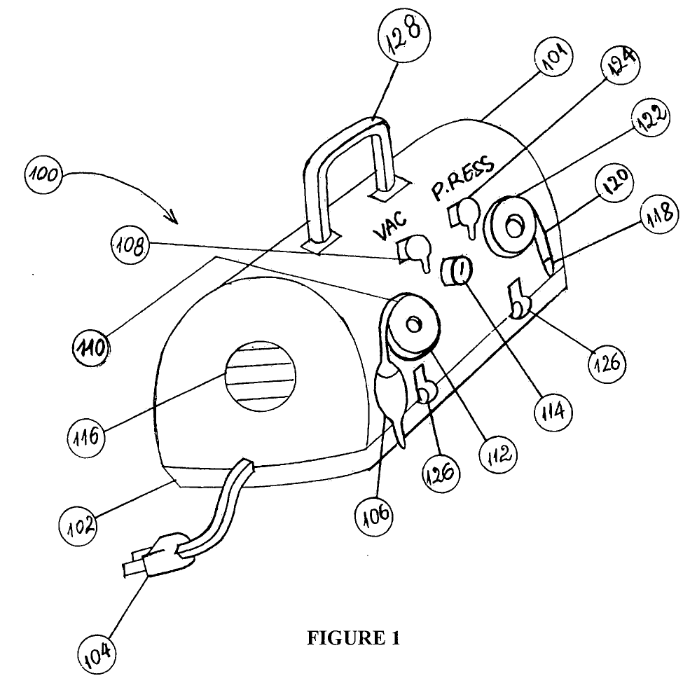

One embodiment of the invention is now described with reference to FIGS. 1 to 6. FIG. 1 shows one embodiment of nasal clearing device 100. Device 100 includes housing 101 attached to base 102, either of which may be formed from metal, plastic, or any other suitable material including bacterial resistant materials. Base 102 may be heavy for durability, noise and vibration reduction, or base 102 may be lightweight for increased portability and ease of handling. Device 100 is sized for hand-held use and ease of transportation and storage.

One method of activating device 100 is to insert power cord 104 into a power outlet, for example a 120 volt alternating current power source. In another embodiment, an ON/OFF switch (not shown) controls power to device 100. In another embodiment, power cord 104 connects to a battery pack (not shown) or is used to charge an internal battery (not shown).

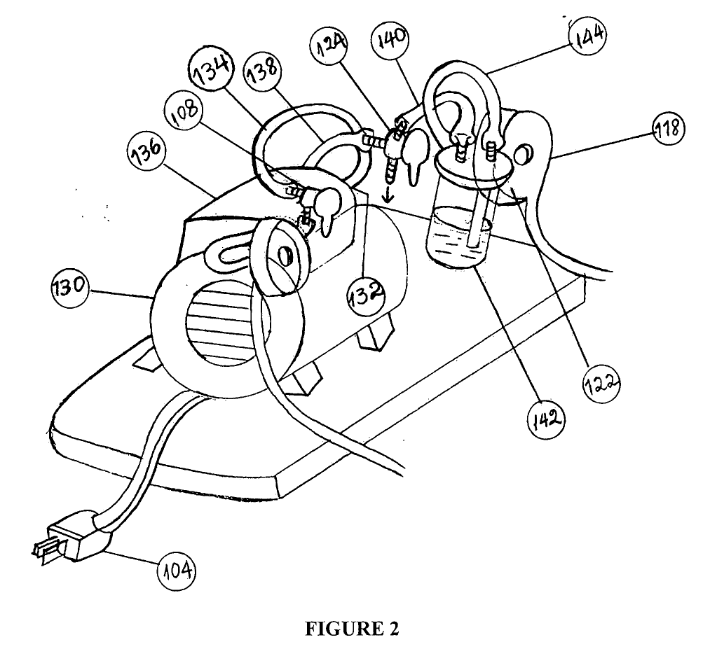

Once device 100 is activated, a vacuum suction is produced at receptacle 106 for removing secretions, for example mucus from an infant's nose. The pressure level of the suction is controlled by valve 108, and may be adjusted, for example, from zero to about 80 cm of mercury. Valve 108 may be, for example, a two-way restrictor valve. Receptacle 106 connects to device 100 with tube 110 that is wound on auto-retracting spool 112. Vacuum gauge 114 provides an indication of the degree of suction, and may provide recommended levels of suction for different degrees of blockage, or children of different ages. Vent 116 in housing 101 provides air for a pump (see FIG. 2), for example for cooling or pumping. In one embodiment device 100 is configured to gradually increase the vacuum pressure if receptacle 106 becomes clogged, up to a safe limit, until the blockage is cleared.

Nozzle 118 is connected to the pump with tube 120 that is wound on auto-retracting spool 122. A liquid reservoir (see FIG. 2) provides pressurized liquid, for example saline, to nozzle 118 for loosening dried or thick secretions prior to or during removal by receptacle 106. Valve 124 controls the pressure to the reservoir and therefore the amount of liquid being directed to nozzle 118. Valve 124 maybe, for example, a three-way diverter valve. Nozzle 118 directs the liquid in either a stream or a spray. Valve 124 varies the pressure to the reservoir between full and off.

Auto-retracting spools 112 and 122 wind up excess tubing for ease of storage and handling. Housing 101 is secured to base 102 with clamps 126. Handle 128 is connected to housing 101 and may be used to transport device 100.

In FIG. 2 there is a diagram illustrating a cutaway view of the nasal clearing device from FIG. 1. The housing 101, auto-retracting spools 112 and 122, receptacle 106 and nozzle 118 have been removed for clarity. Pump 130 is, for example, a transfer pump or any other pump capable of creating a negative air pressure (vacuum) and a positive pressure, and mounted to base 102. In one embodiment, pump 130 is two individual pumps separately creating negative and positive air pressure. Pump 130 is capable of creating pressure up to 25 inches of mercury, both positively and negatively. As previously discussed, pump 130 may operate from alternating or direct current and in one embodiment is powered from batteries, either rechargeable or non-rechargeable. Pump 130 may be selected from commercially available pumps with the qualities of small in size, light in weight, durability, appropriate suction power and low in cost. In one embodiment, pump 130 is constant in speed, voltage and frequency, resulting in a constant output speed, flow, and pressure given a fixed load. In one embodiment, pump 130 is rated at 200 watts and generates flow using piston-type elements resulting in a high dead-head capability. In one embodiment, pump 130 includes an integral filter element and terminates with standard NPT fittings.

Intake port 132 for pump 130 is connected to tube 134. Tube 134 may be, for example, 24-38 French in size and of medical grade. Tube 134 connects to valve 108 and leads to receptacle 106 (see FIG. 1). Intake port 132 provides the vacuum pressure to receptacle 106 during operation.

Output port 136 connects to tube 138 and provides positive air pressure during operation. Tube 138 connects to valve 124. Valve 124 connects to tube 140, which connects to liquid reservoir 142. Reservoir 142 provides liquid through tube 144 to nozzle 118 (see FIG. 1). Referring to both FIGS. 1 and 2, although reservoir 142 is pictured inside housing 101, one of ordinary skill in the art understands that for convenience, sanitation, level-checking of the liquid, reservoir 142 may be located external or internal to housing 101.

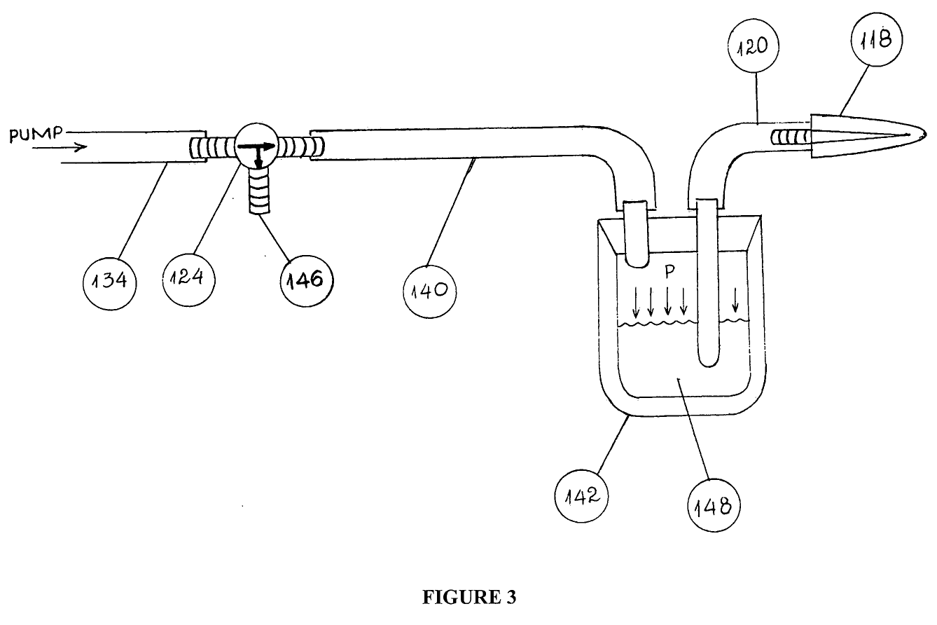

In FIG. 3 there is a diagram illustrating a cross-sectional view of one embodiment of reservoir 142 for use with a nasal clearing device. During operation, pump 130 directs positive air pressure through tube 134 to valve 124. Valve 124 may be manipulated to direct the air either to vent 146, reservoir 142, or split between them, with some air escaping through vent 146 and some air forced into reservoir 142. Vent 146 provides for smoother pump operation while not spraying from nozzle 118. Positive air pressure in reservoir 142 pushes liquid 148, for example a 0.45-0.9% saline mixture, through tube 120 and out of nozzle 118. In one embodiment, nozzle 118 is a mister, size 24-26 French. Nozzle 118 may optionally include a stop (not shown) to limit insertion of nozzle 118 into a nostril.

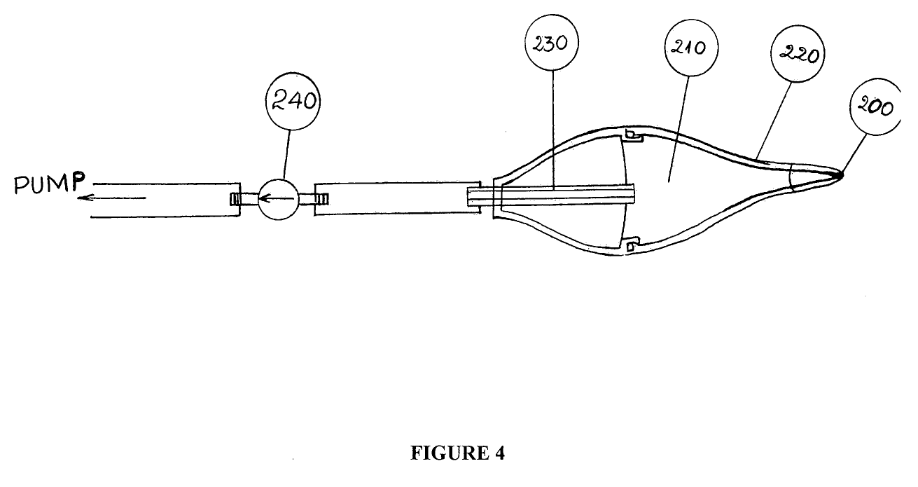

In FIG. 4 there is a diagram illustrating a cross-sectional view of one embodiment of receptacle 106 for use with a nasal clearing device. Receptacle 106 has opening 200 at which the vacuum pressure is created, and into which secretions from a nostril are drawn. Opening 200 is typically less then 5 mm in diameter, and in one embodiment from 3-4 mm in diameter, and may be smaller than the nostril of a typical infant or child. The secretions are drawn into reservoir 210 where the secretions and air separate in order to avoid interfering with continuous vacuum pressure and avoid fouling pump 130.

During use, receptacle 106 is inserted into a nostril up to stop 220. Stop 220 is typically less than 4 mm from opening 200 and has approximately a 12 mm diameter in order to avoid insertion of receptacle 106 too deeply into the nostril of a child or infant. In one embodiment, stop 220 has a minimum diameter of 12 mm, or large enough in order to avoid insertion beyond stop 220 in the average child under three years of age. In one embodiment, stop 220 may form a seal with the nostril in order to improve suction and therefore the removal of secretions. In one embodiment receptacle 106 is sized and shaped for application and sealing at any angle or orientation to a nostril.

After use, receptacle 106 is opened by twisting, pulling, screwing, or otherwise, and may be hinged, threaded, or otherwise secured. Secretions such as mucus are removed and receptacle 106 cleansed. Tube 230 within receptacle maintains the air pressure and is removed from the area of secretion build-up in receptacle 106. Different receptacles may be used with different sizes, shapes and configurations. One of skill in the art will recognize that many variations on receptacle 106 are available and within the scope and coverage of the invention.

Valve 240 is one embodiment of valve 108 from FIG. 1 and is used to control the suction at receptacle 106. The valve used to control suction may be internal or external to the device.

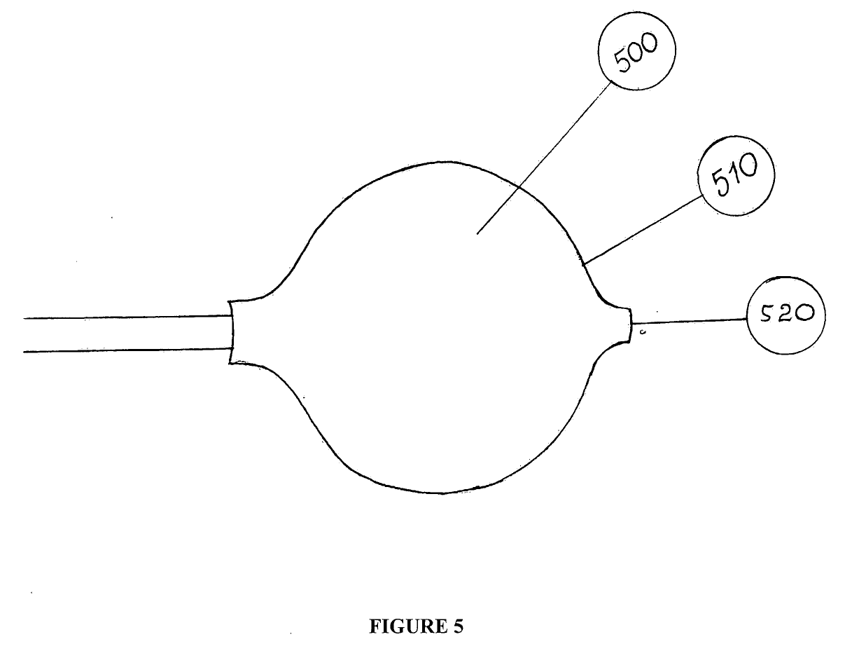

In FIG. 5. there is a diagram illustrating one embodiment of receptacle 106 with bulb 500. Bulb 500 has stop 510, which is designed to be gently pressed against the nostril of a child and form a seal in order to vacuum out secretions. Bulb 500 has opening 520 of less than 5 mm, and in one embodiment approximately 3-4 mm. Stop 510 is less than 4 mm from opening 520, and in one embodiment approximately 2-3 mm. In one embodiment, bulb 500 is sized and shaped to seal against the nostril of a child under the age of three years.

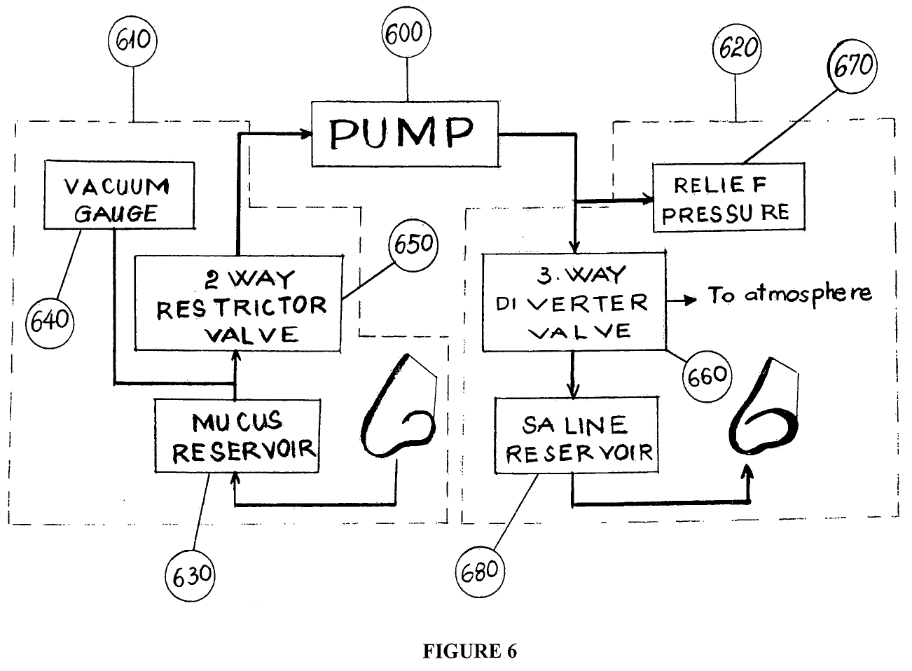

In FIG. 6 there is a block diagram illustrating pressure flow for the nasal clearing device of FIG. 1. The arrows connecting blocks in FIG. 6 illustrate the direction of airflow. Block 600 represents a pump drawing area from section 610 and directing the air to section 620. Consequently, a vacuum, or negative air pressure is developed in the mucus reservoir of section 610 and a positive air pressure is developed in the saline reservoir of section 620. In section 610, air is drawn in from the receptacle, or mucus reservoir, represented by block 630. An optional vacuum gauge, represented by block 640, may indicate the air pressure in section 610 while a valve controls the air pressure. The valve is represented by block 650.

In section 620, air is expelled from the pump and directed through a valve, represented by block 660. There may be an optional relief outlet valve, represented by block 670, to provide an outlet for air from the pump in case of blockage. Optionally there may be a relief inlet valve (not illustrated) in section 610. The air may be directed from the valve to the saline reservoir, represented by block 680.

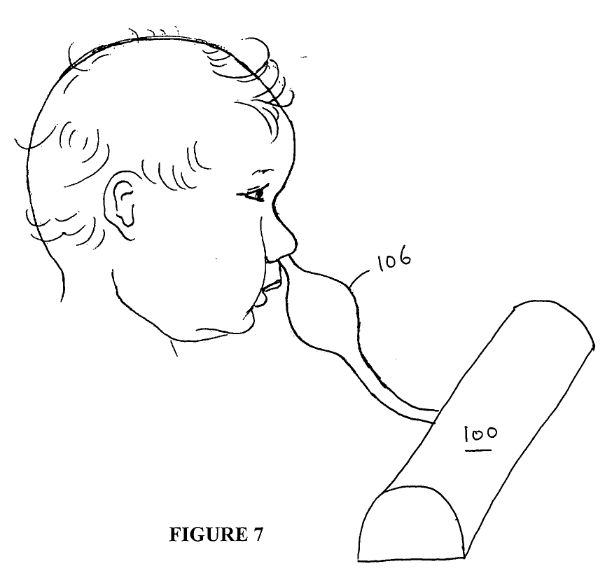

FIG. 7 is a diagram illustrating a perspective view of one embodiment of a bulb and application thereof to a child's nose. Optionally, prior to inserting the opening of the bulb and applying suction, a saline or other liquid mist may be gently sprayed into one or both nostrils in order to loosen dried secretions and aid in their removal. The child may be sitting upright, although application of suction is more easily achieved with the child supine. The child's head may be supported with a pillow and held while the bulb is gently pressed against their nostril to form a seal, thereby improving suction.

A valve is turned to activate and/or adjust suction and suction is typically applied for 5 to 60 seconds to remove mucus. The airway and nostril should be checked after this time period and suction repeated if necessary.

The invention is efficient and rapidly clears secretions from a child's nose, especially when the child has not yet learned to blow their own nose, or is otherwise unable to do so.

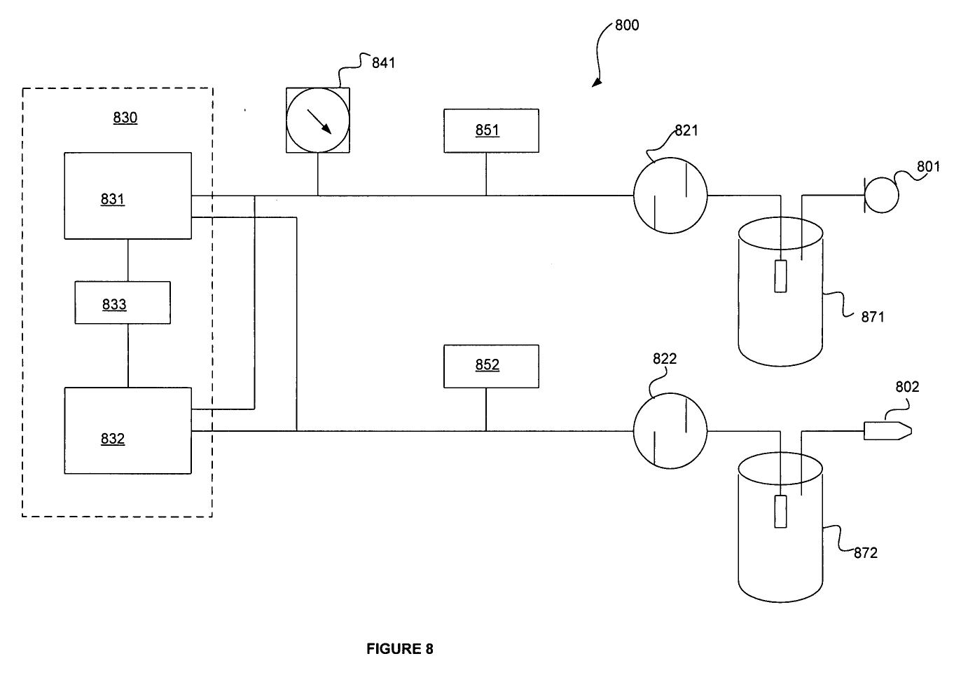

Now referring to FIG. 8, a schematic diagram 800 of the portable device 100 is illustrated. Schematic diagram 800 includes a power driven motor 830 which generates an adjustably continuous vacuuming pressure and an adjustably continuous flushing pressure. Power driven motor 830 further includes a first piston pump 831 and a second piston pump 832. First piston pump 831 is used to generate adjustably continuous vacuuming pressure and second piston pump 832 is used to generate adjustably continuous flushing pressure. Power driven motor 830 also includes a controller 833 electrically connected to first piston pump 831 and second piston pump 832. Controller 833 monitors the temperatures and output pressures of both first piston pump 831 and second piston pump 833. When the temperatures exceed a preset critical temperature, controller 833 will shut down power driven motor 830 as well as first piston pump 831 and second piston pump 832. Similarly, when output pressures exceed a preset pressure value, controller 833 automatically shuts down power driven motor 830. In one embodiment, controller 833 may cause power driven motor 833 to enter a sleep mode where power consumption and currents are minimal when nasal passage cleaning device 800 is not in used.

Referring again to FIG. 8, schematic diagram 800 also includes a first pressure adjusting mean 851 and a second pressure adjusting mean 852. First pressure adjusting mean is designed to adjust the vacuuming pressure output from first piston pump 831 and second pressure adjusting mean is designed to adjust the flushing pressure output from second piston pump 832. In one embodiment, first pressure adjusting means 851 and second pressure adjusting means are mechanical pressure adjustment valves. In one embodiment, first pressure adjusting means 851 and second pressure adjusting means are electrical circuits that can continuously adjust the current of power driven motor 830 so that flushing pressure and vacuuming pressure can be changed accordingly.

Schematic diagram 800 also includes a first air filter 821 connected to a first bottle 8671, a first receptacle 801, and to first piston pump 831 on the vacuuming pressure portion. On the flushing pressure portion, second air filter 822 is connected to a second bottle 872, to second receptacle 802, and to second piston pump 732.

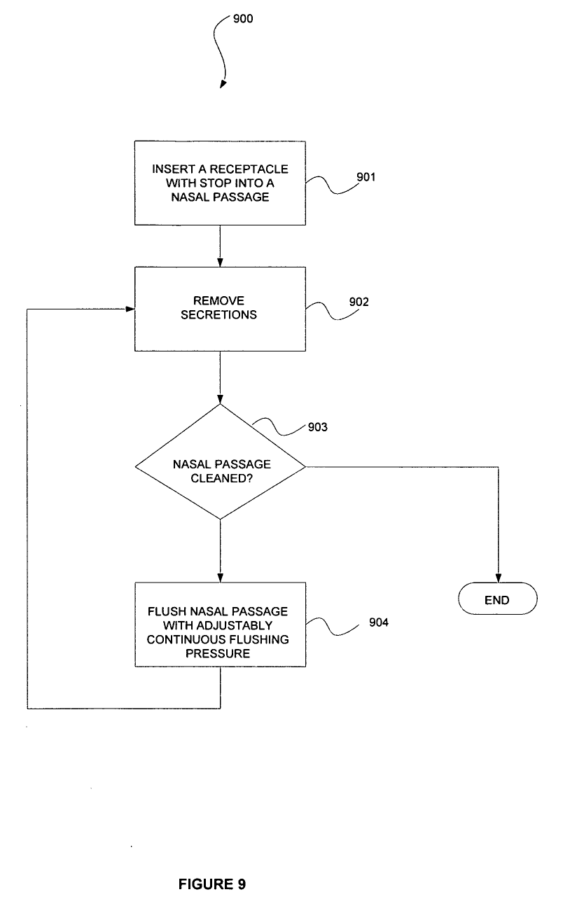

Now referring to FIG. 9, a flow chart diagram 900 illustrating the process of cleaning a nasal passage using a motor driven portable device.

Referring to step 901, inserting a receptacle into a nasal passage by using a stop coupled to said receptacle so that the insertion depth is minimal. Step 901 can be implemented using portable device illustrated in FIG. 100, FIG. 2, and FIG. 8 of the present invention. The first receptacle and stop can be stop 500 as illustrated in FIG. 5.

Referring next to step 902, removing secretions from the nasal passage by applying an adjustably continuous vacuuming pressure generated by a power driven motor. Step 902 can be implemented by a portable device 800 illustrated in FIG. 1, FIG. 2, and FIG. 8.

Referring now to step 903, examining whether the nasal passage has been cleaned. After vacuuming secretions from the nasal passage, the user may visually examine whether the nasal passage is completely cleaned. Other methods of detection can be used such as the sound of the vacuuming pressure.

Finally referring to step 904, if the nasal passage has not been cleaned, flush the nasal passage with a fluid sprayed at adjustably continuous flushing pressure and repeating the steps 901 to 903 above until said nasal passage is cleaned. Step 904 can be implemented by the portable device illustrated in FIG. 1, FIG. 2, and FIG. 8 of the present invention.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. A portable device for cleaning a nasal passage, comprising:
a first receptacle having a first opening and a first stop, mechanically coupled to said first receptacle, said first stop operable to limit the amount of insertion of said first receptacle into said nasal passage;
a second receptacle mechanically coupled to said second receptacle;
a power driven motor, mechanically coupled to said first receptacle and said second receptacle, operable to provide an adjustably continuous flushing pressure at said first receptacle and to provide an adjustably continuous vacuuming pressure at said second receptacle;
a first pressure adjusting means, coupled to said power driven motor and said first receptacle, operable to control the amount of said flushing pressure; and
a second pressure adjusting means, coupled to said motor and said second receptacle, operable to control the amount of said vacuuming pressure.

2. The portable device of claim 1 further comprising a housing means for holding said first receptacle, said second receptacle, said power driven motor, said first pressure adjusting means, and said second pressure adjusting means.

3. The portable device of claim 1 further comprising a controller electrically coupled to said motor, operable to monitor the temperature and pressures of said first receptacle and said second receptacle, wherein when the temperature surpasses a preset critical temperature said controller causes said motor to stop operation, and wherein when said pressures of said first receptacle and said second receptacle surpass preset pressure levels, said controller causes said power driven motor to stop operation.

4. The portable device of claim 1 wherein said first receptacle further comprises a bulb having said first opening therein.

5. The portable device of claim 4 wherein said bulb is widened from said first opening to said first stop and wherein said first stop is configured to form a seal with the nostril of the nose.

6. The portable device of claim 5 wherein said bulb further comprises a reservoir for receiving the secretions from said nasal passage.

7. The portable device of claim 6 wherein said reservoir further comprises a hollow portion of said bulb.

8. The portable device of claim 7 wherein the contours of said first stop have dimensions for sealing against the nostril of a human under the age of 3 years.

9. The portable device of claim 1 wherein said first receptacle further comprises a nozzle extending from said first stop and wherein one end of said first nozzle is in communication with said first opening.

10. The portable device of claim 1 wherein said first pressure adjusting means further comprises a first mechanical valve, and wherein said second pressure adjusting means further comprises a second mechanical valve.

11. The portable device of claim 1 wherein said second receptacle further comprises a nozzle that is coupled to a reservoir.

12. The portable device of claim 11 wherein said nozzle further comprises a mister with a tip measured around 24-26 French.

13. The portable device of claim 1 wherein said first pressure adjusting means further comprises a first electrical bidirectional speed controlling circuit electrically coupled to said motor.

14. The portable device of claim 1 wherein said second pressure adjusting means further comprises a second electrical bidirectional speed controlling circuit electrically coupled to said motor.

15. A portable device for cleaning a nasal passage, comprising:
a receptacle having an opening and a removable stop mechanically coupled to said receptacle, said removable stop operable to limit the amount of insertion of said receptacle into said nasal passage;
a power driven motor, mechanically coupled to said receptacle, operable to provide either an adjustably continuous flushing pressure or to provide an adjustably continuous vacuuming pressure at said receptacle;
a selector, electrically coupled to said motor, operable to allow a user to select between said adjustably continuous flushing pressure or said adjustably continuous vacuuming pressure; and
a pressure adjusting means, coupled to said motor and said receptacle, operable to enable said user to adjust the amount of flushing pressure and the amount of vacuuming pressure.

16. The portable device of claim 15 further comprising a housing means for holding said first receptacle, said power driven motor, said selector, and said pressure adjusting means.

17. The portable device of claim 15 further comprising a controller electrically coupled to said motor, operable to monitor the temperatures and pressures of said receptacle, wherein when the temperature surpasses a preset critical temperature said controller causes said motor to stop operation, and wherein when said flushing or vacuuming pressures of said receptacle surpass a preset pressure level, said controller causes said motor to stop operation.

18. A method of clearing a nasal passage comprising:
inserting a receptacle into said nasal passage by using a stop coupled to said receptacle so that the insertion depth is minimal;
removing secretions from said nasal passage by applying an adjustably continuous vacuuming pressure generated by a power driven motor;
examining whether said nasal passage has been cleaned; and
if said nasal passage has not been cleaned, flushing said nasal passage with a fluid sprayed at adjustably continuous flushing pressure and repeating the steps of inserting a receptacle and removing secretions from said nasal passage until said nasal passage is cleaned.

19. The method of claim 18 wherein said flushing said nasal passage with a fluid sprayed at an adjustably continuous flushing pressure further comprising:
removing said receptacle from said nasal passage;
inserting a second receptacle to said nasal passage; and
applying said adjustably continuous flushing pressure to said second receptacle using said power driven motor controlled by a second pressure adjusting device.

20. The method of claim 18 wherein said removing secretions from said nasal passage further comprises controlling said adjustably continuous vacuuming pressure using a first pressure adjusting device operated by a user.
